SEQUENCE LISTING (1 sequence)

SEQ ID NO: 1 - protein, 289 aa, Human adenovirus 5
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

(19) United States
(12) Patent Application Publication
(10) Pub. No.: US 2022/0136004 A1
(43) Pub. Date: May 5, 2022

(54) Targeted In Vivo Genome Modification

(71) Applicant: Chan Zuckerberg Biohub, Inc., San Francisco, CA (US)

(72) Inventors: Andrew P May, San Francisco, CA (US); Eleonore Tham, San Francisco, CA (US); Lucy Maynard, San Francisco, CA (US); Marco Mignardi, San Francisco, CA (US); Nicole K. Paulk, San Francisco, CA (US); Ryan T. Leenay, San Francisco, CA (US); Gagandeep Renuka Kumar, San Francisco, CA (US)

(21) Appl. No.: 17/422,173

(22) PCT Filed: Jan. 13, 2020
(86) PCT No.: PCT/US20/13403

§ 371 Date: Jul. 9, 2021

Related U.S. Application Data
(60) Provisional application No. 62/791,608, filed on Jan. 11, 2019.

(51) Int. Cl. C12N 15/86; C12N 15/11; C12N 9/22; A61K 48/00; A61K 31/65; A61K 35/15

(52) U.S. Cl. CPC C12N 15/86; C12N 15/11; C12N 9/22; A61K 48/005; A61K 31/65; C12N 2750/14171; C12N 2310/20; C12N 2800/80; C12N 2830/002; C12N 2750/14143; A61K 35/15

(57) ABSTRACT

An in vivo method of modifying a genome of a target cell in a mammalian subject is described. The method includes administering an effective amount of a transducer cell to the subject, where the transducer cell includes a regulated viral vector delivery system (RVVDS) for producing and releasing viral transduction particles (VTPs) when the transducer cell is exposed to inducing conditions, and each VTP comprises a nucleic acid encoding a genome modification system (GMS) comprising a genome modification protein and one or more elements that regulate expression or activity of the genome modification protein in a mammalian cell.

### CROSS-REFERENCE TO RELATED APPLICATIONS

This application claims benefit of U.S. Provisional Application No. 62/791,608 filed Jan. 11, 2019, which is incorporated herein by reference in its entirety.

### GOVERNMENT SUPPORT

This invention was made with U.S. Government support under Grant Number K01-DK107607 (National Institute of Digestive and Diabetes and Kidney Diseases). The U.S. Government has certain rights in the invention.

### FIELD OF THE INVENTION

This invention relates to cell and gene therapy and finds application in the field of medicine.

### BACKGROUND OF THE INVENTION

Systems for targeted genome modification (e.g., “genome engineering”) have great potential in biomedicine. These systems include CRISPR-associated nucleases such as the CRISPR-Cas9 system, zinc-finger nucleases (ZFNs), transcription activator-like effector nucleases (TALENs), homing endonucleases (HEs), meganucleases, megaTAL systems, recombinases, transposases and Cre-lox systems. Viral vectors are the predominant delivery modality used to deliver genome editing systems to primary mammalian cells in vitro and in vivo for genome editing, as other technologies are still in development, such as nanoparticles demonstrated by Lee et al. 2017 “Nanoparticle delivery of Cas9 ribonucleoprotein and donor DNA in vivo induces homology-directed DNA repair,” Nat Biomed Eng. 1:889-901. Example systems for targeted genome modification are described in Chen and Goncalves, 2016, “Engineered Viruses as Genome Editing Devices,” Mol Ther. 24(3):447-57; Dunbar et al., 2018, “Gene therapy comes of age,” Science 359: 6372; and Yang et al., 2016, “A Dual AAV System Enables the Cas9-Mediated Correction of a Metabolic Liver Disease in Newborn Mice,” Nature Biotechnology 34(3):334-38, each incorporated herein by reference. For example, Yang et al., used a hepatotrophic dual-rAAV system to introduce Cas9 and sgRNA into mice to correct a point mutation in the ornithine transcarbamylase gene. Yang et al. achieved gene correction but it occurred at low frequency and with high toxicity. Improved materials and methods for carrying out genome modification are needed.

### BRIEF DESCRIPTION OF THE INVENTION

In one aspect the an isolated mammalian transducer cell is provided, where the cell comprises a regulated viral vector delivery system (RVVDS) for producing and releasing viral transduction particles (VTPs) when the transducer cell is exposed to inducing conditions; and where each VTP comprises a nucleic acid encoding a genome modification system (GMS) comprising a genome modification protein and one or more elements that regulate expression or activity of the genome modification protein in a mammalian cell. In some embodiments. the RVVDS comprises one or more first promoters that control VTP production and release, where at least one first promoter is an inducible promoter, and the one or more elements that regulate expression or activity of the genome modification protein comprise one or more second promoters, where at least one second promoter is an inducible promoter. In some embodiments the at least one second promoter is a cell-specific promoter or a doxycycline-inducible promoter.

In some embodiments, the RVVDS comprises a first vector that is a replication deficient adenovirus vector comprising the GMS and a second vector comprising a nucleic acid sequence encoding an adenovirus E1A protein. In some embodiments the first vector comprises a first promoter and the second vector comprises a second first promoter. In some embodiments the nucleic acid sequence encoding an adenovirus E1A protein does not include an intron(s). In some embodiments the adenovirus E1A protein is a human adenovirus 5 E1A protein comprising an amino acid sequence of SEQ ID NO:1. In some embodiments the cell does not comprise a gene sequence encoding an adenovirus E1B protein. [0007] In some embodiments the genome modification protein may be a nuclease. In some embodiments the GMS comprises an RNA-guided nuclease and the VTP comprises a sequence encoding a nucleic acid targeting moiety. In some cases the genome modification protein is a CRISPR-associated protein (Cas), a Cre recombinase, a zinc-finger nuclease (which may be a Fokl fusion protein), or a transcription activator-like effector nuclease (TALEN). In some embodiments the GMS comprises an RNA-guided nuclease and the VTP comprises a sequence encoding a nucleic acid targeting moiety. For example, The cell may comprise a CRISPR-associated protein, such as Cas9, and a guide RNA.

In some cases, the inducing conditions are proximity to a target cell comprising a cell-surface molecule, and activation of at least one first promoter is mediated by an engineered protein in the transducer cell, where the engineered protein has an extracellular domain that interacts with the cell surface molecule of a target cell and an intracellular signaling domain that activates transcriptional activation from the at least one first promoter in the transducer cell. In some cases, the inducing conditions are exposure of the transducer cell to a small molecule transcriptional activator that activates transcription from at least one first promoter.

In various aspects the transducer cell is a macrophage, lymphocyte, T cell, NK cell, B cell, plasma cell, dendritic cell, neutrophil, eosinophil, basophil, monocyte, or stem cell. In some cases, the cell may be a human cell (e.g., an engineered or modified human cell).

In a related aspect, an in vivo method of modifying a genome of a target cell in a mammalian subject is provided, where the method includes administering an effective amount of a transducer cell to the subject. In a related aspect, an in vivo method of modifying a genome of a target cell in a mammalian subject is provided, where the method includes administering an effective amount of a transducer cell, wherein the regulated viral vector delivery system is activated by contact between the transducer cell and the target cell, by proximity of the transducer cell to the target cell, or by co-administration of the transducer cell and an regulating agent to the subject, and the second promoter is active, or becomes active, in the target cell. In some cases the VTP comprises a nucleic acid targeting sequence with homology to a target nucleic acid sequence in the genome of the target cell.

In a related aspect, the invention provides a mammalian cell that comprises a first polynucleotide encoding a replication defective adenovirus vector comprising a genetic cargo and a second polynucleotide that encodes an Adenovirus E1A protein with the proviso that the cell does not comprise an Adenovirus E1B protein or a polynucleotide that encodes an adenovirus E1B protein.

### BRIEF DESCRIPTION OF THE DRAWINGS

FIG. 1 illustrates a transducer cell that delivers viral transduction particles (VTPs) to a target cell. Upon activation of the regulated viral vector delivery system (RVVDS) in the transducer cell, VTPs containing or encoding a genome modification system (GMS) are produced, released, and delivered to the target cell. In the target cell, components of the GMS are expressed and the GMS can then effect a desired genome modification in the target cell. In FIG. 1, the RVVDS (and therefore VTP production) is shown as regulated by an exogenous small molecule and/or synNotch receptor, and the GMS is shown as a CRISPR-Cas9 system. As will be clear from the following discussion, FIG. 1 is provided to illustrate certain aspects of the invention but is not intended to be limiting.

FIG. 2 shows in vivo dosing of empty transducer cells expressing Firefly luciferase. Dilutions of 2×10⁶ RAW264.7 cells were injected via tail vein into albion C57/B6 mice and imaged over multiple days. Signal was localized in the liver, and possible lymph nodes.

### DETAILED DESCRIPTION OF THE INVENTION

This invention is a novel platform to perform in vivo genome modification, circumventing issues such as liver uptake and immunogenicity. A transducer cell, carrying a latent viral payload, can circumvent the immune response and translocate to regions of tissue that may be unreachable with current systems using purified viral vectors administered intravenously.

### 1. INTRODUCTION

Disclosed herein is an in vivo method of modifying a genome of a mammalian target cell through administration of a population of mammalian transducer cells to a mammalian subject. In some approaches the genome sequence is modified (e.g., by changing the nucleic acid sequence of a specific target sequence in the genome). In some approaches, the genome is modified epigenetically (e.g., the methylation pattern at a specific target sequence in the genome is changed).

Transducer cells are recombinantly engineered mammalian cells that contain a regulated viral vector delivery system (RVVDS) for producing “viral transduction particle(s)” or VTP(s). The RVVDS comprises components (DNA, RNA and proteins) that, when expressed (e.g., transcribed and/or translated) or activated, cause the transducer cell to produce and release VTPs. Transducer cells may be prepared by introducing an RVVDS into a “pre-transducer cell,” such as an autologous cell obtained from a patient to be treated or progeny of such a cell). Typically the RVVDS includes multiple components that may be separately introduced into the cell. In one approach two components are introduced to produce the transducer cell: A first vector which is sometimes a replication defective viral backbone that lacks the E1 gene and sometimes also lacks the E3 and E4 genes, as discussed below in Section 4.1.1.1). In this approach the first vector delivers the GMS components (e.g., comprising a cargo encoding elements of the GMS) along with components required for VTP production (e.g., capsid proteins, packaging proteins, and other components needed for VTP production). The second component may be a vector for delivering a replication/transcriptional activator for the virus, such as the Adenovirus E1 protein. As noted elsewhere herein, in one approach the E1 protein is the E1A protein encoded by a minimal gene, generally without the E1B encoding sequence. See Section 4.1.1.2 and Example 1, below. Thus, in some embodiments the first vector is derived from a replication-defective viral vector (e.g., delta-E1 Adenovirus) and the second vector provides elements required for viral replication (e.g., a nucleic acid sequence encoding a transcriptional activator such as Adenovirus E1 protein).

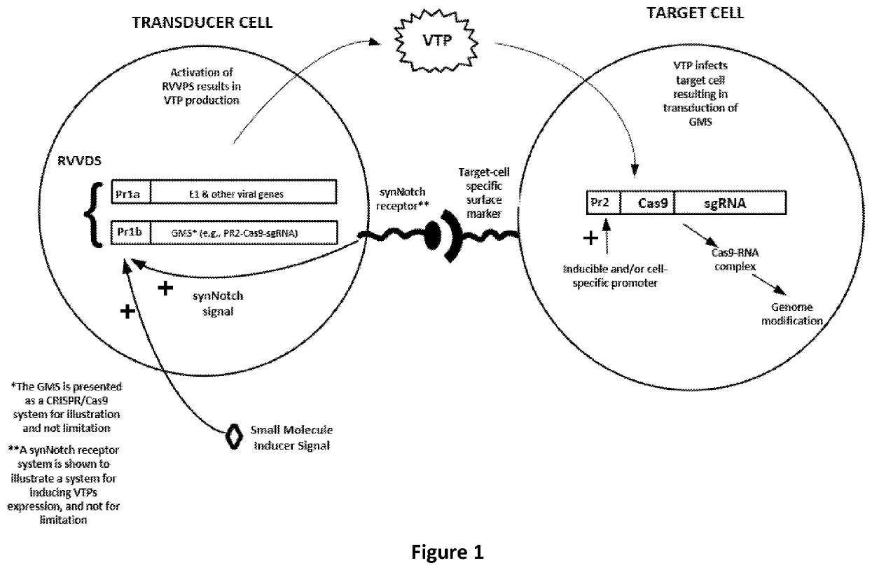

Expression or activity of the RVVDS is controlled such that, when a transducer cell is in an inducing environment (i.e., exposed to inducing conditions), VTPs are produced and released. VTPs may be released from a transducer cell by budding, exocytosis, or cell lysis, or by other mechanisms). Where the RVVDS comprises multiple components each component may be independently expressed. For example, transcription from one component may be under control of a constitutive promoter, and transcription from another component may be under control of an inducible promoter. For example, referring to FIG. 1, Promoter 1a (Pr1a) could be constitutive and Promoter 1b (Pr1b) could be inducible. It will be understood that expression or activation of at least one component of the RVVDS will be regulated (e.g., under control of an inducible promoter) so that production of VTPs is regulated and can be induced when the cell is exposed to inducing conditions. In some cases, expression or activation of multiple RVVDS components is inducible, and each of the multiple components may be induced by the same or different agents or conditions. For example, the regulatory apparatus for different components (e.g., Pr1a and Pr1b in FIG. 1) may be the same or different.

Production and release of the VTPs by the transducer cell results in infection of target cells, thereby introducing genome modification system (GMS) components into the target cells. In one approach, at the time of VTP release, the transducer cell and the target cell are in sufficient proximity that such VTP transfer can occur rapidly, for example by direct diffusion of viral particles from the transducer cell to the target cell. See FIG. 1.

Examples of an inducing environment are proximity of a transducer cell to a target cell, or presence of a detectable signal within a local environment or niche in which the transducer cell locates or can migrate to. Another example of an inducing environment occurs when the transducer cell is exposed to a chemical transcriptional activator (e.g., a small molecule that is not cell-bound) that interacts directly or indirectly with the first promoter(s) to activate expression of the RVVDS in the transducer cell. One example of an inducing environment is contact between the transducer cell and the target cell. In one approach, for example and not limitation, a cell surface receptor protein of the transducer cell may contact a cell surface ligand on the target cell, and the contact activates VTP production by the RVVDS. For example, the contacting may activate expression of RNA or proteins encoded in the RVVDS, including elements of the VTP genome (i.e., ‘cargo’). In one approach, activation is mediated by an engineered protein having an extracellular domain that binds or interacts with a cell surface molecule of a target cell and an intracellular signaling domain that mediates transcriptional activation.

The VTPs comprise a cargo (called “a genome modification system” or “GMS”) that enables genome modification in a target cell. For illustration and not limitation, a GMS may be a CRISPR-Cas system, TALEN, Cre-lox, or ZFN-based GMS, or any engineered versions therein, e.g., as described in greater detail below. The GMS generally includes one or more a regulatory element to control expression of the genome modification apparatus in the target cell. As used herein, and as will be apparent from context, “genome modification system” can refer to a nucleic acid cargo of the VTP and/or polypeptides and RNAs encoded by the VTP cargo. As used herein, “cargo” or “genetic cargo” has its normal meaning in the gene therapy art, i.e., a polynucleotide(s) delivered to a cell by a gene therapy vector.

In one approach, the VTP comprises a nucleic acid component(s) (which may be RNA, DNA, or modified derivatives thereof) that encodes a genome modification protein. In one approach the genome modification protein is a nuclease (e.g., CAS, Cre recombinase, or a Fok1 endonuclease fused to a Zinc Finger or a TALE effector). See, e.g., Makarova, et al., 2018, “Classification and Nomenclature of CRISPR-Cas Systems: Where from Here? The CRISPR Journal 1:5, describing CRISPR-Cas systems. The VTP may also comprise a component, or targeting moiety, that directs the genome modifying protein to a specific target sequence within the target cell. In one approach the targeting moiety is a nucleic acid comprising a sequence with homology to the specific target sequence within the target cell. In the specific, but not limiting case of DNA components, expression of at least some GMS components in the target cell is under control of at least one promoter (“GMS promoter” or “second promoter”), e.g., operably linked to the nuclease-encoding sequence. For illustration, exemplary targeting moieties include, a TALE DNA-binding domain, ZF DNA-binding domain, sgRNA, or DNA encoding sgRNA and a promoter operably linked to the sgRNA encoding sequence. The GMS promoter may be an inducible promoter or cell-specific promoter (see Section 6, below). In some cases the GMS promoter is a cell-specific promoter where the cell specificity corresponds to the target cell.

In another approach, the VTP comprises an engineered version of a viral vector, where GMS components (including non-DNA components) are packaged into VTPs through alternative mechanisms (e.g., mRNA recruitment, protein fusions, protein-protein binding). These virus-like particles, or VLPs, can be used to deliver alternative cargos compared to other viral vectors, such as mRNA or already translated genome modifying proteins. For purposes of this disclosure, reference to a VTP encompasses VLPs, except where otherwise specified or clear from context. In one approach the VTP is a VLP containing a fusion protein with a DNA binding domain and a transcriptional activator domain. In one approach the DNA binding domain is derived from a CAS protein.

Typically a single VTP comprises or encodes all exogenous components required for genome modification, whether that be in DNA, RNA, protein, or any combination thereof. However, dual or multiple component VTPs are also contemplated in which, for example, the genome modifying protein and targeting moieties are introduced in separate VTPs. VTPs may also comprise capsid proteins or other molecules required for either effective transducibility or genomic engineering of the target cell. As used herein, ‘transducibility’ refers to the ability of a VTP to transduce its cargo to a target cell.

In the following sections aspects of the invention are described in additional detail.

### 2. TRANSDUCER CELL AND TARGET CELL

2.1 Transducer Cell

The transducer cell is a genetically engineered cell that acts as a means to produce the genome modification components in a host, and ultimately the target cell. Generally, the transducer cell and target cell are mammalian, e.g., mouse, rat, primate or human. Usually the transducer cell and target cell are from the same species, and in some cases, the transducer cell and target cell are autologous (i.e., the target cell is from a subject and the transducer cell is an engineered cell derived from a cell obtained from the subject). In some embodiments, the transducer cell preferentially migrates to, or is retained in, the vicinity of the target cells. For example a transducer cell may be a macrophage that migrates to inflamed organ tissue (e.g. lung) containing the target cells. In alternative embodiments, the position of the transducer cell is fixed and either the VTPs translocate to the target cell or the target cell translocates to the proximity of the transducer cell. In some embodiments, the both the target cell and transducer cell are motile.

The transducer cell can be any cell capable of expressing the RVVDS. In one approach the transducer cell is motile cell and/or circulating cell, such as a macrophage, lymphocyte, T cell, NK cell, B cell, plasma cell, dendritic cell, neutrophil, eosinophil, basophil, monocyte, stem cell or similarly motile but engineered cells.

2.2 Target Cell

The target cell can be any nucleated cell for which modification of the genomic DNA is desired. Exemplary target cells include hepatocytes, neurons, myocytes, retinal cells, hematopoietic cells, stem cells, cancer cells. Examples of target cells are listed in Table 3 of U.S. Pat. No. 6,475,789, incorporated herein, and shown in TABLE 1:

TABLE 1
Target cells where the genomic DNA can be modified by this invention

```
Keratinizing Epithelial Cells
keratinocyte of epidermis (differentiating epidermal cell)
basal cell of epidermis (stem cell)
keratinocyte of fingernails and toenails
basal cell of nail bed (stem cell)
hair shaft cells
  medullary, cortical, cuticular; hair-root sheath
  cells, cuticular, of Huxley’s layer, of Henle’s layer
  external; hair matrix cell (stem cell)
Cells of Wet Stratified Barrier Epithelia
surface epithelial cell of stratified squamous epithelium
of tongue, oral cavity, esophagus, anal canal, distal
urethra, vagina
basal cell of these epithelia (stem cell)
cell of external corneal epithelium
cell of urinary epithelium (lining bladder and urinary ducts)
Epithelial Cells Specialized for Exocrine Secretion
cells of salivary gland
  mucous cell (secretion rich in polysaccharide)
  serous cell (secretion rich in glycoprotein enzymes)
  cell of von Ebner’s gland in tongue (secretion to
  wash over taste buds)
cell of mammary gland, secreting milk
cell of lacrimal gland, secreting tears
cell of ceruminous gland of ear, secreting wax
cell of eccrine sweat gland, secreting glycoproteins (dark cell)
cell of eccrine sweat gland, secreting small molecules (clear cell)
cell of apocrine sweat gland (odoriferous secretion,
sex-hormone sensitive)
cell of gland of Moll in eyelid (specialized sweat gland)
cell of sebaceous gland, secreting lipid-rich sebum
cell of Bowman’s gland in nose (secretion to wash over
olfactory epithelium)
cell of Brunner’s gland in duodenum, secreting alkaline
solution of mucus and enzymes
cell of seminal vesicle, secreting components of seminal
fluid, including fructose (as fuel for swimming sperm)
cell of prostate gland, secreting other components of
seminal fluid
cell of bulbourethral gland, secreting mucus
cell of Bartholin’s gland, secreting vaginal lubricant
cell of gland of Littré, secreting mucus
cell of endometrium of uterus, secreting mainly
carbohydrates
isolated goblet cell of respiratory and digestive tracts,
secreting mucus
mucous cell of lining of stomach
zymogenic cell of gastric gland, secreting pepsinogen
oxyntic cell of gastric gland, secreting HCI
acinar cell of pancreas, secreting digestive enzymes and
bicarbonate
Paneth cell of small intestine, secreting lysozyme
type II pneumocyte of lung, secreting surfactant
Clara cell of lung
Cells specialized for Secretion of Hormones
cells of anterior pituitary, secreting
      growth hormone, follicle-stimulating hormone,
      luteinizing hormone, prolactin, adrenocorticotropic
      hormone, and thyroid-stimulating hormone,
cell of intermediate pituitary, secreting
  melanocyte-stimulating hormone
cells of posterior pituitary, secreting
  oxytocin, vasopressin
cells of gut, secreting
  serotonin, endorphin, somatostatin, gastrin,
  secretin, cholecystokinin, insulin and glucagon
cells of thyroid gland, secreting
  thyroid hormone, calcitonin
cells of parathyroid gland, secreting
  parathyroid hormone, oxyphil cell
cells of adrenal gland, secreting
  epinephrine, norepinephrine, and steroid hormones;
  mineralocorticoids
  glucocorticoids
cells of gonads, secreting
  testosterone (Leydig cell of testis)
  estrogen (theca intema cell of ovarian follicle)
  progesterone (corpus luteum cell of ruptured ovarian follicle)
cells of juxtaglomerular apparatus of kidney
  juxtaglomerular cell (secreting renin)
macula densa cell
peripolar cell
mesangial cell
Epithelial Absorptive Cells in Gut, Exocrine Glands, and Urogenital Tract
brush border cell of intestine (with microvilli)
striated duct cell of exocrine glands
gall bladder epithelial cell
brush border cell of proximal tubule of kidney
distal tubule cell of kidney
nonciliated cell of ductulus efferens
epididymal principal cell
epididymal basal cell
Cells Specialized for Metabolism and Storage
hepatocyte (liver cell)
fat cells
  white fat
  brown fat
  lipocyte of liver
Epithelial Cells Serving Primarily a Barrier Function, Lining the Lung,
Gut, Exocrine Glands, and Urogenital Tract
type I pneumocyte (lining air space of lung)
pancreatic duct cell (centroacinar cell)
nonstriated duct cell of sweat gland, salivary gland,
mammary gland
parietal cell of kidney glomerulus
podocyte of kidney giornerulus
cell of thin segment of loop of Henle (in kidney)
collecting duct cell (in kidney)
duct cell of seminal vesicle; prostate gland
Epithelial Cells Lining Closed Internal Body Cavities
vascular endothelial cells of blood vessels and lymphatics
  fenestrated
  continuous
  splenic
synovial cell (lining joint cavities; secreting largely hyaluronic acid)
serosal cell (lining peritoneal, pleural, and pericardial cavities)
squamous cell lining perilymphatic space of ear
cells lining endolymphatic space of ear
  squamous cell
  columnar cells of endolymphatic sac
  with microvilli
  without microvilli
  “dark” cell
  vestibular membrane cell (resembling choroid plexus cell)
  stria vascularis basal cell
  stria vascularis marginal cell
  cell of Claudius
  cell of Boettcher
choroid plexus cell (secreting cerebrospinal fluid)
squamous cell of pia-arachnoid
cells of ciliary epithelium of eye
  pigmented
  nonpigmented
corneal “endothelial” cell
Ciliated Cells with Propulsive Function
of respiratory tract
of oviduct and of endometrium of uterus (in female)
of rete testis and ductulus efferens (in male)
of central nervous system (ependymal cell lining brain cavities)
Cells Specialized for Secretion of Extracellular Matrix
epithelial:
  ameloblast (secreting enamel of tooth)
  plenum sernilunaturn cell of vestibular apparatus of ear
  (secreting proteoglycan)
  interdental cell of organ of Corti (secreting tectorial
  “membrane” covering hair cells of organ of Corti)
nonepithelial (connective tissue)
  fibroblasts (various-of loose connective tissue, of
cornea, of tendon, of reticular tissue of bone marrow, etc.)
  pericyte of blood capillary
  nucleus pulposus cell of intervertebral disc
  cementoblast/cementocyte (secreting bonelike cernenturn of
  root of tooth)
  odontoblastiodontocyte (secreting dentin of tooth)
  chondrocytes
  of hyaline cartilage, of fibrocartilage, of elastic cartilage
  osteoblast/osteocyte
  osteoprogenitor cell (stem cell of osteoblasts)
  hyalocyte of vitreous body of eye
  stellate cell of perilymphatic space of ear
Contractile Cells
skeletal muscle cells
    red (slow)
    white (fast)
    intermediate
    muscle spindle-nuclear bag
    muscle spindle-nuclear chain
    satellite cell (stem cell)
heart muscle cells
    ordinary
    nodal
    Purkinje fiber
smooth muscle cells
myoepithelial cells
    of iris
    of exocrine glands
Cells of Blood and Immune System
red blood cell
megakaryocyte
macrophages
    monocyte
    connective tissue macrophage (various)
    Langerhans cell (in epidermis)
    osteoclast (in bone)
    dendritic cell (in lymphoid tissues)
    microglial cell (in central nervous system)
neutrophil
eosinophil
basophil
mast cell
T lymphocyte
    helper T cell
    suppressor T cell
    killer T cell
B lymphocyte
    IgM
    lgG
    lgA
    IgE
killer cell
stem cells for the blood and immune system (various)
Sensory Transducers
photoreceptors
    rod
    cones
    blue sensitive
    green sensitive
    red sensitive
Hearing
    inner hair cell of organ of Corti
    outer hair cell of organ of Corti
acceleration and gravity
    type I hair cell of vestibular apparatus of ear
    type II hair cell of vestibular apparatus of ear
taste
    type 11 taste bud cell
smell
    olfactory neuron
    basal cell of olfactory epithelium (stem cell for
    olfactory neurons)
blood Ph
    carotid body cell
    type I
    type II
touch
    Merkel cell of epidermis
    primary sensory neurons specialized for touch
temperature
    primary sensory neurons specialized for temperature
    cold sensitive
    heat sensitive
pain
  primary sensory neurons specialized for pain
configurations and forces in musculoskeletal system
  proprioceptive primary sensory neurons
Autonomic Neurons
cholinergic
adrenergic
peptidergic
Supporting Cells of Sense Organs and of Peripheral Neurons
supporting cells of organ of Corti
    inner pillar cell
    outer pillar cell
    inner phalangeal cell
    outer phalangeal cell
    border cell
    Hensen cell
supporting cell of vestibular apparatus
supporting cell of taste bud (type I taste bud cell)
supporting cell of olfactory epithelium
Schwann cell
satellite cell (encapsulating peripheral nerve cell bodies)
enteric glial cell
Neurons and Glial Cells of Central Nervous System
neurons
glial cells
    astrocyte
    oligociendrocyte
Lens Cells
anterior lens epithelial cell
lens fiber (crystallin-containing cell)
Pigment Cells
melanocyte
retinal pigmented epithelial cell
Germ Cells
oogonium/oocyte
spermatocyte
spermatogonium (stem cell for spermatocyte)
Nurse Cells
ovarian follicle cells
Sertoli cell (in testis)
thymus epithelial cell
Stem Cells
embryonic stem cell
embryonic germ cell
    adult stem cell
    fetal stem cell
```

2.3 Target Cell/Transducer Cell Combinations

In some embodiments, therapeutic transducer cells of the invention are administered to a patient using a route that allows for at least some transducer cells to migrate to a location proximal to target cells and/or to physically interact with target cells. VTPs or the target cell can also perform the migratory or interaction function. In one approach transducer cells are selected based on naturally occurring cell-cell or cell-tissue interactions with the target cells or tissue. For example, dendritic cells naturally are located near exterior contact barriers such as the skin. Accordingly, if a target cell is within the epidermis, a dendritic cell could be selected as the transducer cell. Similarly, if the target cell is a blood cell, a T lymphocyte could be selected as the transducer cell type (e.g., CAR-T cell targeting a B-cell malignancy through an anti-CD19 mechanism). Alternatively, or in addition, chemical gradients can be used to direct transducer cells to a desired location, utilizing known recruitment migration mechanisms such as those reviewed by Huaqing Cai and Peter N. Devreotes 2011, in “Moving in the right direction: How eukaryotic cells migrate along chemical gradients” Semin Cell Dev Biol. 22(8): 834-841.

2.4 Transducer Cell Administration

Transducer cells may be administered to a subject using any suitable route of administration known in the art. For example, the cells may be inoculated parenterally (including, for example, intravenous, intraperitoneal, intramuscular, intradermal, and subcutaneous), by ingestion, or by application to mucosal surfaces. The transducer cells of the invention can be administered locally by direct injection into a tissue containing, or leading to target cells. Routes of administration include epidermal administration including subcutaneous or intradermal injections. Transdermal transmission including iontophoresis may be used, for example “patches” that deliver product continuously over periods of time. Mucosal administration of the engineered cells of the invention is also contemplated, including intranasal administration with inhalation of aerosol suspensions. Suppositories and topical preparations may also be used.

### 3. GENOME MODIFICATION SYSTEMS AND TARGETING NUCLEIC ACID SEQUENCES

Genome modification systems are well known in the art, including CRISPR-associated nucleases (also known as RNA-guided nucleases or RGNs) such as CRISPR-Cas9 nucleases, Zinc-finger nucleases (ZFNs), transcription activator-like effector nucleases (TALENs), Homing endonucleases, Meganucleases; Cre-lox (Cre-induced recombination between cryptic loxP sites), and engineered versions of each thereof. See, e.g., Sander et al., 2014, “CRISPR-Cas systems for editing, regulating and targeting genomes,’ Nat Biotechnol. 32(4):347-55; Urnov et al., 2010, “Genome editing with engineered zinc finger nucleases,” Nat Rev Genet. 11(9):636-46; Sun et al., 2013, “Transcription activator-like effector nucleases (TALENs): a highly efficient and versatile tool for genome editing,” Biotechnol Bioeng. 110(7):1811-21; Sengupta et al., 2017, “Viral Cre-LoxP tools aid genome modification in mammalian cells” J. Biological Engineering 11:45, (describing lentivirus and adeno associated virus delivery systems for Cre-Lox) and Nagy 2000, “Cre recombinase: the universal reagent for genome tailoring,” Genesis, 26(2):99-109, each incorporated by reference herein. In each genome modification method, a specific nucleic acid sequence is targeted, and a subsequent modification is made. These modifications include the target sequence being edited by homologous recombination, non-homologous end joining, homology-directed repair, histone modification, transcriptional activation, RNA editing, transcriptional repression, or other processes that modify the target cell using a GMS. The process by which a specific nucleic acid sequence is targeted varies with the system used. For example, two alternative, but not limiting, examples of engineering without cleavage are described by Thakore et al. 2018 in “RNA-guided transcriptional silencing in vivo with S. aureus CRISPR-Cas9 repressors” Nat Commun. 9(1):1674 and by Amabile et al. 2016 in “Inheritable Silencing of Endogenous Genes by Hit-and-Run Targeted Epigenetic Editing” Cell 167(1): 219-232.e14, where modification was performed on the target genome.

In one approach the VTP cargo component includes (i) a sequence encoding a protein with a nuclease function (i.e., a genome editing nuclease) and (ii) a “targeting nucleic acid sequence.” The nature of the targeting nucleic acid sequence may vary, as is illustrated in TABLE 2, below, which is provided for illustration and not limitation. For example, the targeting nucleic acid sequence may be an RNA (e.g., sgRNA), a DNA sequence encoding an RNA, a DNA sequence encoding a protein or domain of a fusion protein (e.g., TALE DNA-binding domain), or another component(s) that affect sequence specificity of the nuclease. The function carried out by the targeting nucleic acid sequence (whether carried out by the VTP genome nucleic acid directly, or by an encoded RNA or peptide sequence) may be referred to as “directing genome modification in a target cell.”

TABLE 2

| | Exemplary Genome Modification Protein | |
|---|---|---|
| MODE | Encoded By VTP Genome | Nucleic Acid Targeting Moiety |
| CRISPR | DNA encoding a Cas9 protein | sgRNA |
| ZEN | DNA encoding Engineered Fok1 endonuclease | DNA encoding zinc finger |
| | fused to Zinc Finger | DNA-binding domain |
| TALEN | DNA encoding Engineered Fok1 endonuclease | DNA encoding TALE |
| | DNA cleavage domain fused to TAL effector | DNA-binding domain |
| Cre-Lox | DNA encoding a Cre recombinase | LoxP DNA targeting sequence |

### 4. VIRAL VECTOR DELIVERY SYSTEMS (RVVDS)

In conventional viral vector gene therapy methods, virions carrying cargo are generated in producer cells and purified prior to administration to patients or subject and various methods are known for producing viral vectors for gene therapy or other uses. A person of ordinary skill in the art of gene therapy, guided by this disclosure, will be able to adapt elements from these methods when making viral vector delivery systems for use in accord with the present invention. See, e.g., Broussau et al., 2008, “Inducible Packaging Cells for Large-scale Production of Lentiviral Vectors in Serum-free Suspension Culture” Mol. Ther. 16(3):500-7; Luo et al., 2007, “A protocol for rapid generation of recombinant adenoviruses using the AdEasy system” Nat Protoc. 2(5):1236-47; Jager et al., 2009, “A rapid protocol for construction and production of high-capacity adenoviral vectors,” Nature Protocols, 4(4), 547-564, Aponte-Ubillus et al., 2018, “Molecular design for recombinant adeno-associated virus (rAAV) vector production,” Appl Microbiol Biotechnol. 102(3):1045-1054, Goins et al. 2008, “Construction and production of recombinant herpes simplex virus vectors,” Methods Mol Biol. 433:97-113; and Sengupta et al., 2017, “Viral Cre-LoxP tools aid genome modification in mammalian cells” J. BiologicalEngineering 11:45, each of which is incorporated by reference herein. Also see, e.g., Grieger et al., 2002, “Production of Recombinant Adeno-associated Virus Vectors Using Suspension HEK293 Cells and Continuous Harvest of Vector From the Culture Media for GMP FIX and FLT1 Clinical Vector” Mol Ther. 24(2): 287-297.; Penaud-Budloo et al., 2018., “Pharmacology Of Recombinant Adeno-Associated Virus Production” Molecular Therapy: Methods & Clinical Development 8:166-180; each of which is incorporated by reference herein.

4.1 Viral Vectors

Several viral vector systems are known and suitable for making VTPs in the present invention. For illustration and not limitation, systems for use in the present invention include, but are not limited to, lentivirus, adenovirus (AdV), recombinant adenovirus (rAdV), recombinant adeno-associated virus (rAAV), pox virus, alphavirus, retrovirus, arenavirus, measles, rabies, coronavirus, and herpes virus-based systems, or engineered, infectious versions thereof (e.g., VLPs). Each vector system is well-understood by those of skill in the art. For general reviews see Keeler et al., 2017, “Gene Therapy 2017: Progress and Future Directions” Clin Trans/Sci 10(4):242-248, Naso et al., 2017, “Adeno-Associated Virus (AAV) As A Vector For Gene Therapy” BioDrugs 31:317; Dunbar et al., 2018, “Gene therapy comes of age,” Science 359: 6372, incorporated herein by reference. Other viral vectors, or engineered versions thereof, known in the art may be used. In particular, adenovirus (AdV), adeno-associated virus (AAV) and lentivirus-based vectors (each of which is discussed in greater detail below and in the references cited herein) may be used in the practice of the invention.

Viral vectors may be selected based on the nature of the target cell. For example, many serotypes of AAV are known, which differ in the types of cells they infect, as reviewed by Wu et al. 2006, “Adeno-associated virus serotypes: vector toolkit for human gene therapy” Mol Ther. 14(3):316-27. In addition, the species, cell and tissue tropism of viral vectors can be manipulated by genetic engineering various means known to those in the art. See, e.g., Castle et al., 2016, “Controlling AAV Tropism in the Nervous System with Natural and Engineered Capsids” Methods in Molecular Biology (Clifton, N.J.) 1382: 133-149; Cronin et al., 2005, “Altering the Tropism of Lentiviral Vectors through Pseudotyping” Current gene therapy 5.4: 387-398; Paulk, et al., 2018, “Bioengineered AAV capsids with combined high human liver transduction and unique humoral seroreactivity” Molecular Therapy 26(1): 289-303, “Paulk, et al., 2018. Bioengineered viral platform for intramuscular passive vaccine delivery to human skeletal muscle” Molecular Therapy Methods & Clinical Development 10: 144-155, “Engineering of Adenovirus Vectors Containing Heterologous Peptide Sequences in the C Terminus of Capsid Protein IX” J. Virol, 76(14): 6893-6899; incorporated herein by reference.

4.1.1 Adenovirus

In some embodiments, a vector derived from adenovirus is used as the viral vector delivery system. AdV (or recombinant human adenovirus rAdV) systems are known in the art. See e.g., William S. M. Wold1 and Karoly Toth 2015, “Adenovirus Vectors for Gene Therapy, Vaccination and Cancer Gene Therapy” Curr Gene Ther. 13(6): 421-433, incorporated herein by reference. Adenoviruses are double stranded DNA viruses with 36 kb linear genomes with Inverted Terminal Repeat (“ITR”) sequences on either end, as well as a cis-regulatory packaging signal termed “psi” (Alba, Bosch, & Chillon, 2005). Adenoviruses typically remain episomal in the host cell, but on occasion will integrate into the host genome (Giacca & Zacchigna, 2012). Adenoviruses have been employed as vectors for both gene therapy and genome engineering, and can infect dividing or non-dividing cells using a variety of receptors (Sharma et al., 2010; Appaiahgari & Vrati, 2015; Giacca & Zacchigna, 2012; Shim et al., 2017). For example, several clinical trials are underway to assess adenoviral vectors delivering ZFNs to cells ex vivo as a treatment for HIV infection (Shim et al., 2017; Yin, Kauffman, & Anderson, 2017).

4.1.1.1 Replication Defective Adenovirus Vectors

In some embodiments, recombinant adenoviral vectors comprise the adenoviral genome with the viral gene E1 deleted to prevent the vector from being replication competent (Giacca & Zacchigna, 2012). E3 and E4 are also commonly deleted to allow for more adenoviral genomic space for transgenes. For example, one commonly used plasmid backbone for recombinant adenovirus (pAdEasy-1) comprises the genome of adenovirus serotype type-5 (“Ad5”), with the E1 and E3 genes deleted (He, Zhou, Da Costa, Kinzler, & Vogelstein, 1998). In place of the deleted viral genes, one can introduce a gene or genes of interest for packaging and transduction by the adenoviral vector. In adenoviral vectors in which E1 alone is deleted, the gene or genes of interest may be up to 5.1 kb in length, and in vectors in which both E1 and E3 are deleted (e.g., pAdEasy-1), the inserted gene or genes may be up to 8.3 kb in length (Giacca & Zacchigna, 2012).

The E1 gene product is necessary for adenoviral replication, and, when using vectors such as pAdEasy-1 in which E1 is deleted, E1 must be supplied in trans by a host packaging cell. For example, E1-transformed human embryonic kidney cells known as HEK293 cells, among others, may be used as adenoviral vector packaging cells (He et al., 1998; Kovesdi & Hedley, 2010). In some embodiments, a recombinant adenoviral vector with the viral genes E1, E3, and E4 deleted is used (e.g., pAdEasy-2), allowing for the insertion of a gene or genes of interest up to 8.6 kb in length (He et al., 1998). In these embodiments, a host packaging cell must supply both the E1 and E4 genes (e.g., 911E4 cells, in which E4 expression is regulated by an inducible promoter) (He et al., 1998). Plasmids, protocols, and packaging cells for the production of adenoviral vectors are readily available, for example, from the non-profit plasmid repository Addgene. See addgene.org/viral-vectors/adenovirus/; coloncancer.org/adeasy/protocol2.htm), as are many other vendors such as Agilent (agilent.com/en/product/protein-expression/protein-expression-vectors-kits/viral-mediated-delivery-systems/adeasy-adenoviral-vector-systems-232997). (Domain names are provided herein without the http, https, or www prefix; persons of ordinary skill in the art will understand how to append http, https, or www to produce the complete domain names.) To regulate the viral production for this invention, latency can be introduced by regulating expression of the necessary E1 or E4 proteins, as shown previously (Muthana et al. 2011).

In some embodiments, so-called “gutless,” “gutted,” “helper-dependent,” or “high-capacity” adenoviral vectors are used as gene transfer vectors (Alba et al., 2005; Giacca & Zacchigna, 2012; Kovesdi & Hedley, 2010). In these embodiments, all viral proteins necessary for replication and capsid formation are expressed from a “helper plasmid”, and only the gene or genes of interest are flanked by the ITRs and the psi packaging signal and thus competent for packaging by the viral vector (Alba et al., 2005). This allows for gene or genes of interest to be up to approximately 37 kb in length (Giacca & Zacchigna, 2012). Protocols for constructing and producing high-capacity adenoviral vectors are readily available (Jager et al., 2009; Palmer & Ng, 2003). For example, a high-capacity adenoviral vector was used to deliver Cas9 and multiple guideRNAs encoded on a single vector to human primary cells (Ehrke-Schulz et al., 2017). Similar strategies can be adapted to the transducer cell by those knowledgeable about the art.

4.1.1.2 Use of AdV E1A Protein for Superior Replication Switch

In one approach for the present invention, a replication defective adenoviral vector in which the E1 gene has been deleted is used to deliver the GMS cargo. As discussed above, generally some or all other viral genes are deleted as well. As discussed above it is necessary, in such systems, to provide the E1 protein in trans and in certain embodiments of the present invention, E1 is provided in trans in the transducer cell. In one approach, E1 is provided by cotransfecting (or otherwise introducing) a cell with a viral vector carrying the GMS cargo and a viral or plasmid vector encoding an AdV E1 protein. As described in EXAMPLE 1, below, we have discovered that, surprisingly, superior AdV replication is achieved in a cell that expresses AdV E1A protein, both not the E1B protein.

The Adenovirus E1 gene enables an adenovirus replication switch. The E1 gene comprises 3074 nucleotides and encodes two proteins, called E1A and E1B. E1A enables viral genome replication by regulating cell cycle. E1A activates transcription of a number of viral genes as well as genes of the host cell resulting in stimulation of the cell from G1 to S phase to drive quiescent cells into the cell cycle. This enables the virus to use cellular DNA replication machinery for viral genome replication. The native E1A gene for Human adenovirus 5 comprises one intron and encodes at least 3 isoforms via alternatively spliced transcripts (32 kDa, 26 kDa and 6 kDa). The E1A gene sequence for Human adenovirus 5 is found at uniprot.org/uniprot/P03255. The protein sequence for the human AdV5 is provided below:

### Sequence of E1a Protein of Human Adv5

```
[SEQ ID NO: 1]
                10 20 30 40
MRHIICHGGV ITEEMAASLL DQLIEEVLAD NLPPPSHFEP
                50 60 70 80
PTLHELYDLD VTAPEDPNEE AVSQIFPDSV MLAVQEGIDL
                90 100 110 120
LTFPPAPGSP EPPHLSRQPE QPEQRALGPV SMPNLVPEVI
              130 140 150 160
DLTCHEAGFP PSDDEDEEGE EFVLDYVEHP GHGCRSCHTH
              170 180 190 200
RRNTGDPDIM CSLCYMRTCG MFVYSPVSEP EPEPEPEPEP
              210 220 230 240
ARPTPRPKMA PAILRRPTSP VSRECNSSTD SCDSGPSNTP
              250 260 270 280
PEIHPVVPLC PIKPVAVRVG GRRQAVECIE DLLNEPGQPL
DLSCKRPRP
```

E1B prevents cellular inhibition of viral genome replication by suppressing apoptosis. The E1B gene encodes at least 5 isoforms via alternatively spliced transcripts (55 kDa, 19 kDa, 18 kDa, 16 kDa, 15 kDa). E1B 55K: binds to and inactivates the transcriptional regulator p53, thus blocking transcription of genes normally activated by p53 and contributing to the suppression of apoptosis. The E1B 19K isoform suppresses apoptosis by mimicking the action of cellular protein Bcl-2.

As described in EXAMPLE 1, below, we have discovered that, surprisingly, superior AdV replication is achieved in a cell that expresses AdV E1A protein, both not the E1B protein. In one aspect of the invention, the transducer cell produces VTPs derived from an Adenovirus (e.g., a human Adenovirus, such as human Adenovirus 5) and the transducer cell expresses AdV E1A protein, and does not express AdV E1B protein.

In one embodiment the E1A protein is from the human Adenovirus 5 and has the sequence of SEQ ID NO:1. In one approach the E1A protein is expressed from a intronless gene. In one approach the E1A protein is expressed from a coding sequence less than 1 kB in length, sometimes less than 0.9 KB in length. In one approach the E1A protein is from human Adv 5. In one approach the E1A protein is from a human AdV related embodiments selected from the following list. In this list “HAdV” means “human Adenovirus”; “-xxx” (e.g., “-C1”, “-05”) identifies the virus type, and the virus designation is followed by the GenBank accession number for the virus genome. Persons of ordinary skill in the art can readily identify the E1A protein coding sequence for each virus.
HAdV-C1 AC_000017; HAdV-C2 AC_000007; HAdV-B3 AY599834; HAdV-E4 AY599837; HAdV-05 AY601635; HAdV-C6 FJ349096; HAdV-B7 KP670856.2; HAdV-D8 AB448767; HAdV-D9 AJ854486; HAdV-D10 J N226746; HAdV-B11 AF532578; HAdV-A12 X73487; HAdV-D13 J N226747; HAdV-B14 J0824845; HAdV-D15 KF268204; HAdV-B16 J N860680; HAdV-D17 H0910407; HAdV-A18 GU191019; HAdV-D19 J0326209; HAdV-D20 J N226749; HAdV-1321 AY601633; HAdV-D22 FJ619037; HAdV-D23 J N226750; HAdV-D24 J N226751; HAdV-D25 J N226752; HAdV-D26 EF 153474; HAdV-D27 J N226753; HAdV-D28 FJ824826; HAdV-D29 J N226754; HAdV-D30 J N226755; HAdV-A31 AM 749299; HAdV-D32 JN226756; HAdV-D33 J N226758; HAdV-1334 AY737797; HAdV-B35 AC_000019; HAdV-D36 GQ384080; HAdV-037 AB448775; HAdV-D38 J N226759; HAdV-D39 J N226760; HAdV-F40 NC_001454; HAdV-F41 DQ315364.2; HAdV-042 J N226761; HAdV-D43 J N226762; HAdV-D44 J N226763; HAdV-D45 J N226764; HAdV-D46 AY875648; HAdV-D47 J N226757; HAdV-D48 EF 153473; HAdV-D49 0Q393829; HAdV-B50 AY737798; HAdV-D51 J N226765

See Ismail et al., 2018, “Adenoviromics: Mining the Human Adenovirus Species D Genome,” Front. Microbiol. 9:2178.

In some embodiments the E1A protein has the sequence of the corresponding naturally occurring (wild-type) Adenovirus. However, it will be recognized that some variation from the naturally occurring sequence is permitted without negatively affecting viral replication. For example, in some embodiments the transducer cell comprises an E1A protein with less than 100% identity to a naturally occurring protein, for example, at least 80% identity, at least 90% identity or at least 95% identity.

4.1.2 Adeno-Associated Virus

In some embodiments, a vector derived from Adeno-associated virus (AAV), a member of the Parvovirus family, is a small nonenveloped, icosahedral virus with single-stranded linear DNA genomes of 4.7 kilobases (kb). rAAV (e.g., recombinant adeno-associated viral vector) systems are known in the art. See, e.g., Naso et al., 2017, “Adeno-Associated Virus (AAV) as a Vector for Gene Therapy” BioDrugs. 31(4): 317-334, incorporated herein by reference. AAV is assigned to the genus, Dependovirus, because the virus was discovered as a contaminant in purified adenovirus stocks (D. M. Knipe, P. M. Howley, Field's Virology., Lippincott Williams & Wilkins, Philadelphia, ed. Sixth, 2013). In its wild-type state, AAV depends on a helper virus—typically adenovirus—to provide necessary protein factors for replication, as AAV is naturally replication-defective. The 4.7-kb genome of AAV is flanked by two inverted terminal repeats (ITRs) that fold into a hairpin shape important for replication. Being naturally replication-defective and capable of transducer nearly every cell type in the human body, AAV represents an ideal vector for therapeutic use in gene therapy or vaccine delivery. In its wild-type state, AAV's life cycle includes a latent phase during which AAV genomes, after infection, are site specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains (A. Vasileva, R. Jessberger, Nature reviews. Microbiology, 3:837-847 (2005)). Rep and Cap are then replaced with an array of possible transfer vector configurations to perform gene addition or gene targeting. These vectorized recombinant AAVs (rAAV) transduce both dividing and non-dividing cells, and show robust stable expression in quiescent tissues. The properties of non-pathogenicity, broad host range of infectivity, including non-dividing cells, and potential site-specific chromosomal integration make AAV an attractive tool for gene transfer and genomic engineering.

The number of rAAV gene therapy clinical trials that have been completed or are ongoing to treat various inherited or acquired diseases is increasing dramatically as rAAV-based therapies increase in popularity. Similarly, in the clinical vaccine space, there have been numerous recent preclinical studies and ongoing clinical trials using rAAV as a vector to deliver antibody expression cassettes in passive vaccine approaches for human/simian immunodeficiency virus (HIV/SIV), influenza virus, henipavirus, and human papilloma virus (HPV). (See, Johnson et al. 2009, Balazs et al. 2012, Balazs et al. 2014, Balazs et al. 2013, Limberis et al. 2013, Limberis et al. 2013, Sipo et al. 2011, Ploquin et al. 2013, Kuck et al. 2006, Nieto et al 2009, Nieto et al. 2012, and Zhou et al. 2010)

Plasmids for producing AAV that encode the i) replication and pseudotyping packaging functions; ii) helper functions; iii) and the ITR-containing transfer vector, described above, are readily commercially available, for example, from the nonprofit plasmid repository Addgene (addgene.org/viral-vectors/aav/), CellBioLabs (cellbiolabs.com), and other vendors. These viral vectors can also be used for virus-like particle (VLP) formation, utilizing only the necessary parts of the viral proteins (in the example but not limiting case, using VP3) (Hoque et al. 1999, Zeltins 2013).

Because the AAV genome is small (4.7 kb including the ITRs, approximately 4.5 kb excluding the ITRs) and some gene editing nuclease genes are relatively large (for example, the Streptococcus pyogenes Cas9 gene is approximately 4.2 kb), various strategies have been developed to circumvent this size limitation problem. For example, dual AAV vectors have been used to separately encapsulate one construct containing a Cas9 gene flanked by ITRs, and a second construct containing the expression components necessary for sgRNA expression, flanked by ITRs. A non-limiting example was demonstrated by Thakore et al. in 2018 when they used a dual CRISPR genome modification system to modify cholesterol levels. In addition, smaller Cas9 orthologs have been identified that allow for the combination of Cas9 and sgRNA sequences into a single AAV vector particle (Ran et al., 2015). Any combination of these known in the art can be used in the transducer cell, where the GMS is packaged within the “transfer vector” and the “packaging” and “helper” functions are provided by the transducer cell.

4.1.3 Lentivirus

In some embodiments a vector derived from a lentivirus is used as the viral vector delivery system. Lentiviruses are retroviruses, single stranded RNA viruses that undergo reverse transcription into DNA and integrate into the host genome (Sakuma, Barry, & Ikeda, 2012). Lentivirus (e.g., VSV-G coated lentiviral vector) systems are known in the art. See e.g., Sakuma et al. 2012, “Lentiviral vectors: basic to translational,” Biochem J. 443(3):603-18 incorporated herein by reference. Lentiviral vectors for both gene therapy and genome modification have been derived from the lentivirus human immunodeficiency virus (“HIV”) (Cockrell & Kafri, 2007; Sakuma et al., 2012; Shim et al., 2017). The HIV genome is an approximately 9 kb linear genome that encodes three structural genes (gag, pol and env), two regulatory genes (rev and tat), and four accessory genes (vif, vpr, vpu and nef) (Sakuma et al., 2012). The genes are flanked by long terminal repeats (“LTRs”) that are necessary for viral transcription, reverse transcription, and integration, and there is also a cis-regulatory sequence, termed “psi,” that is necessary for packaging of the genome into the viral capsid (Sakuma et al., 2012).

Lentiviral vectors can infect both dividing and non-dividing host cells. In some embodiments, lentiviral vectors are “pseudotyped” by the replacement of the env gene with the gene encoding the vesicular stomatitis virus envelope glycoprotein (“VSV-G”), a rhabdoviral attachment protein that allows for entry of the pseudotyped lentivirus into a broader range of cell types (Cronin et al. 2006, Sakuma et al., 2012). Packaging cell lines have been used to produce lentiviral virions for gene therapy and gene editing, which are later purified and used to treat primary cells or dose directly into patients. A packaging cell line expresses two “packaging” genetic constructs that encodes gag and pol and rev, an “envelope” genetic construct that encodes an envelope protein (e.g., VSV-G), and a “transfer” genetic construct that comprises one or more genes of interest flanked by the LTRs, and further comprises the psi cis-regulatory packaging signal (Sakuma et al., 2012). The gene or genes of interest encoded on the transfer plasmid may be up to approximately 8.5 kb in length. A cell may be transformed into a packaging cell, for example, by transfection of the cell with packaging, envelope and transfer plasmids. Protocols for generating lentiviral packaging cell lines are readily available (Merten et al. 2016). Lentiviral packaging vectors have gone through several “generations” of plasmid schemes as improvements in safety of using lentiviral vectors in a human subject have been developed (Sakuma et al., 2012). For example, in second-generation lentiviral vectors a single packaging plasmid encodes gag, pol, tat and rev, with separate envelope and transfer plasmids as described above (Sakuma et al., 2012). In third-generation lentiviral vectors the packaging plasmid is divided such that one plasmid encodes gag and pol, and a second plasmid encodes rev (Sakuma et al., 2012). In addition, a promoter that is independent of Tat is added to the transfer plasmid to promote the expression of the gene or genes of interest in a way that does not require the Tat protein. Plasmids for the generation of lentiviral packaging vectors are readily available, for example, from the nonprofit plasmid repository Addgene (addgene.org/viral-vectors/lentivirus/), Cell Biolabs (cellbiolabs.com/lentiviral-complete-expression-systems), or Thermo Fisher (thermofisher.com/us/en/home/references/protocols/proteins-expression-isolation-and-analysis/protein-expression-protocol/lentiviral-expression- systems.html).

In some embodiments, engineered versions of lentiviral vectors have been created. Integrase-defective lentivirus vectors (“IDLVs”) are one example, where specific mutations introduced into the integrase protein product such that a delivered transfer genetic construct will not integrate into the genome, and therefore will not be stably maintained (Banasik & McCray, 2010). An additional, but not limiting example is that of virus-like particles, or VLPs, generated from lentiviral components. In these vectors, a fraction of genes are expressed (e.g., VSV-G) and genome modification components are recruited into the VLP (Montagna et al. 2018).

Lentiviral vectors have been used to deliver genome modification proteins including Cas9, zinc finger nucleases (ZFNs), and transcription activator-like effector nucleases (TALENs) (Cai, Bak, & Mikkelsen, 2014; Shim et al., 2017). For example, one study used a lentivirus to deliver DNA encoding Cas9 as well as four guideRNAs on one lentiviral transfer genetic construct (Kabadi, Ousterout, Hilton, & Gersbach, 2014). Another study used a library of lentiviruses to then generate a library of tens of thousands of guideRNAs to conduct a genetic screen in human cells (Wang, Wei, Sabatini, & Lander, 2014).

4.1.4 References Describing Viral Vector

Alba, R., Bosch, A., & Chillon, M. (2005). Gutless adenovirus: last-generation adenovirus for gene therapy. Gene Therapy, 12 Suppl 1(S1), S18-27; Appaiahgari, M. B., & Vrati, S. (2015). Adenoviruses as gene/vaccine delivery vectors: promises and pitfalls. Expert Opinion on Biological Therapy, 15(3), 337-351; Ehrke-Schulz, E., et al., 2017. “CRISPR/Cas9 delivery with one single adenoviral vector devoid of all viral genes” Nature Publishing Group, 7(1), 17113; Giacca, M., & Zacchigna, S. (2012). Virus-mediated gene delivery for human gene therapy. Journal of Controlled Release, 161(2), 377-388. He, T.-C., Zhou, S., Da Costa, L. T., Kinzler, K. W., & Vogelstein, B. (1998). A simplified system for generating recombinant adenoviruses. Proceedings of the National Academy of Sciences of the United States of America, 1-6; Jager, L., Hausl, M. A., Rauschhuber, C., Wolf, N. M., Kay, M. A., & Ehrhardt, A. (2009). A rapid protocol for construction and production of high-capacity adenoviral vectors. Nature Protocols, 4(4), 547-564; Kovesdi, I., & Hedley, S. J. (2010). Adenoviral producer cells. Viruses, 2(8), 1681-1703; Palmer, D., & Ng, P. (2003). Improved system for helper-dependent adenoviral vector production. Molecular Therapy, 8(5), 846-852; Muthana, M et al. (2011). Use of macrophages to target therapeutic adenovirus to human prostate tumors. Cancer Res. 2011 Mar. 1; 71(5):1805-15; Sharma, A., Li, X., Bangari, D., & Mittal, S. 2009. Adenovirus receptors and their implications in gene delivery. Virus Res. August; 143(2): 184-194; Shim, G., Kim, D., Park, G. T., Jin, H., Suh, S.-K., & Oh, Y.-K. (2017). Therapeutic gene editing: delivery and regulatory perspectives. Acta Pharmacologica Sinica, 38(6), 738-753; Yin, H., Kauffman, K. J., & Anderson, D. G. (2017). Delivery technologies for genome editing. Nature Reviews. Drug Discovery, 16(6), 387-399; Knipe, D. M., Howley, P. M., Field's Virology., Lippincott Williams & Wilkins, Philadelphia, 6^{th} ed., 2013 Vasileva, A., Jessberger, R., 2005, Precise hit: adeno-associated virus in gene targeting. Nature Reviews Microbiology 3, 837-847; Johnson, P. R., et al, 2009. Vector-mediated gene transfer engenders long-lived neutralizing activity and protection against SIV infection in monkeys. Nature medicine 15, 901-906; Balazs, A. B., et al, 2012. Antibody-based Protection Against HIV Infection by Vectored ImmunoProphylaxis Nature 481, 81-84; Balazs, A. B., et al, 2014. Vectored immunoprophylaxis protects humanized mice from mucosal HIV transmission. Nature Medicine 20, 296-300; Balazs, A. B., et al, 2013. Broad protection against influenza infection by vectored immunoprophylaxis in mice. Nature Biotechnology 31, 647-652; Limberis, M. P., et al, 2013. Intranasal antibody gene transfer in mice and ferrets elicits broad protection against pandemic influenza. Science Translational Medicine 5, 187ra172; Limberis, M. P., et al, 2013. Vectored Expression of the Broadly Neutralizing Antibody F16 in Mouse Airway Provides Partial Protection against a New Avian Influenza A Virus, H7N9. Clinical and Vaccine Immunology: CVI 20, 1836-1837; Sipo, I., et al, 2011. Vaccine protection against lethal homologous and heterologous challenge using recombinant AAV vectors expressing codon-optimized genes from pandemic swine origin influenza virus (SOIV). Vaccine 29, 1690-1699; Ploquin, A., et al, 2013. Protection against henipavirus infection by use of recombinant adeno-associated virus-vector vaccines. The Journal of Infectious Diseases 207, 469-478; Kuck, D., et al, 2006. Intranasal vaccination with recombinant adeno-associated virus type 5 against human papillomavirus type 16 L1. Journal of Virology 80, 2621-2630; Nieto, K., et al, 2009, Combined prophylactic and therapeutic intranasal vaccination against human papillomavirus type-16 using different adeno-associated virus serotype vectors. Antiviral therapy 14, 1125-1137; Nieto, K., et al, 2012, Intranasal vaccination with AAV5 and 9 vectors against human papillomavirus type 16 in rhesus macaques. Human gene therapy 23, 733-741; Zhou, L., et al, 2010. Long-term protection against human papillomavirus e7-positive tumor by a single vaccination of adeno-associated virus vectors encoding a fusion protein of inactivated e7 of human papillomavirus 16/18 and heat shock protein 70. Human gene therapy 21, 109-119; Ran, F. A., et al, 2015. In vivo genome editing using Staphylococcus aureus Cas9. Nature 520, 186-191; Thakore, P. I., et al, 2018. RNA-guided transcriptional silencing in vivo with S. aureus CRISPR-Cas9 repressors. Nature Communications 9, 1674; Lino et al. (2018). Delivering CRISPR: a review of the challenges and approaches. Drug Delivery, 25(1), 1234-1257; Banasik, M. B., & McCray, P. B. (2010). Integrase-defective lentiviral vectors: progress and applications. Gene Therapy, 17(2), 150-157; Cai, Y., Bak, R. O., & Mikkelsen, J. G. (2014). Targeted genome editing by lentiviral protein transduction of zinc-finger and TAL-effector nucleases. eLife, 3, e01911; Cockrell, A. S., & Kafri, T. (2007). Gene delivery by lentivirus vectors. Molecular Biotechnology, 36(3), 184-204; Cotmore, S. F., & Tattersall, P. (2014). Parvoviruses: Small Does Not Mean Simple. Annual Review of Virology, 1(1), 517-537; Cronin, J., Zhang, X-Y, & Reiser J. (2005). Altering the Tropism of Lentiviral Vectors through Pseudotyping. Curr Gene Ther. 2005 August; 5(4): 387-398; Kabadi, A. M., Ousterout, D. G., Hilton, I. B., & Gersbach, C. A. (2014). Multiplex CRISPR/Cas9-based genome modification from a single lentiviral vector. Nucleic Acids Research, 42(19), e147-e147; Kotterman, M. A., & Schaffer, D. V. (2014). Engineering adeno-associated viruses for clinical gene therapy. Nature Reviews. Genetics, 15(7), 445-451. doi.org/10.1038/nrg3742; Lau, C.-H., & Suh, Y. (2017). In vivo genome editing in animals using AAV-CRISPR system: applications to translational research of human disease. F1000Research, 6, 2153; Merten, O.-W. et al. (2016) Production of lentiviral vectors. Mol Ther Methods Clin Dev. 2016; 3: 16017; Montagna, C. et al. (2018) VSV-G-Enveloped Vesicles for Traceless Delivery of CRISPR-Cas9. Mol. Ther. Nucleic Acids 12, 453-462; Ran, F. A., Cong, L., Yan, W. X., Scott, D. A., Gootenberg, J. S., Kriz, A. J., et al. (2015). In vivo genome editing using Staphylococcus aureus Cas9. Nature, 520(7546), 186-191; Sakuma, T., Barry, M. A., & Ikeda, Y. (2012). Lentiviral vectors: basic to translational. Biochemical Journal, 443(3), 603-618; Senis, E., Fatouros, C., Große, S., Wiedtke, E., Niopek, D., Mueller, A.-K., et al. (2014). CRISPR/Cas9-mediated genome engineering: An adeno-associated viral (AAV) vector toolbox. Biotechnology Journal, 9(11), 1402-1412Wang et al., 2014, “Genetic Screens in Human Cells Using the CRISPR-Cas9 System,” Science 343:80-84.

### 6. REGULATION OF RVVDS AND GMS

6.1 Regulation Generally

The expression in the transducer cell of the RVVDS can be under control of an inducible promoter system, sometimes referred to as the “first promoter.” In addition, the expression in the target cell of viral vector sequences encoding GMS components is under control of a promoter system, which may be constitutive or inducible and/or cell-specific, sometimes referred to as the “second promoter.” The first and second promoter systems can have different properties and functions.

Additional regulatory elements, such as DNA binding proteins for transcriptional regulation, microRNAs, translational regulators, protein regulation through degradation tags, and others can also be used to regulate expression (at the DNA, RNA, or protein level as reviewed by Kitada et al. 2018 “Programming gene and engineered-cell therapies with synthetic biology,” Science 359(6376)) in either the transducer cell or the target cell.

As used herein the term “promoter” is understood to refer broadly to sequences that control transcription or the rate of transcription. As is well known in the art, regulation of gene expression may involve, in addition to a minimal promoter (a short DNA sequence comprised of a TATA-box and other sequences that serve to specify the site of transcription initiation), various regulatory elements including enhancers, terminator sequences, polyadenylation sequences, and the like. In particular, reference to a “promoter” herein, unless otherwise indicated by context, may include an associated enhancer sequence or other regulatory sequences that affect expression (e.g., transcription, translation, splicing, etc.). Promoters and other regulatory elements, such as enhancers, are “operably linked” to a nucleic acid sequence when they affect to the expression of RNA from the nucleic acid sequence. A promoter and an enhancer are “associated” with each other when they are operably linked to the same gene sequence.

Promoters may be constitutive (e.g., in a particular cell type), or regulatable. Promoters, whether constitutive or regulatable, may function in a cell-specific manner. A specific, but not limiting example, is that of the sgRNA in the CRISPR GMS. The Pol III system is required to drive transcription (Such as U6, H1, tRNA-based) of sgRNAs as demonstrated by Cong et al., 2013, “Multiplex Genome Engineering Using CRISPR/Cas Systems”, Science. 339(6121): 819-823; and Mefferd et al., 2015, “Expression of CRISPR/Cas single guide RNAs using small tRNA promoters”, RNA. 21(9): 1683-1689. Pol III systems can be used to drive genome modifying protein expression, in which some can be regulated and some cannot. Tissue-specific promoters are also well known in the art as explained by K. Saukkonen and A. Hemminki, 2004, in “Tissue-specific promoters for cancer gene therapy”, Expert Opin Biol Ther. 4(5):683-96; which can be used to as “promoter 2” for additional regulation. As used herein, a tissue-specific promoter can be considered a cell-specific promoter. In the context of this disclosure, reference to an inducible promoter is intended to encompass a cell-specific promoter (i.e., a promoter that is induced by cellular factors).

Transcription is regulated by transcription factors, which are typically DNA binding proteins that bind to enhancer or promoter elements. Regulation of transcription may involve recruitment or modification of transcription factors, disruptions of repressor binding to DNA, and other processes. See Mullick et al., 2006, “The cumate gene-switch: a system for regulated expression in mammalian cells,” BMC Biotechnology 6:43. As used here, except as otherwise clear from context, references to “regulation,” “regulator” and the like should be understood as any process that alters transcription, without limitation to a particular mechanism.

Other methods of regulation also exist to change expression, or activity of proteins either alongside, or independent of, promoters. This includes examples, but not limited to, DNA regulation using methylation demonstrated by Amabile et al. 2016 in “Inheritable Silencing of Endogenous Genes by Hit-and-Run Targeted Epigenetic Editing” Cell, 167(1): 219-232.e14, or de-methylation as shown by Xu et al. 2016 in “A CRISPR-based approach for targeted DNA demethylation” Cell Discov. 2:16009. Regulation also can be performed at the RNA level, as shown by (but not limited to) Xie et al. 2011 in “Multi-input RNAi-based logic circuit for identification of specific cancer cells” Science. 333(6047):1307-11, where microRNAs were used in a posttranscriptional circuit to trigger a cellular response. Regulation at the protein level is also known by those in the art, shown by (but not limited to) Banaszynski et al. 2006 in “A rapid, reversible, and tunable method to regulate protein function in living cells using synthetic small molecules” Cell 126(5):995-1004 where small molecules were used to regulate protein activity.

6.2. Regulated Viral Vector Delivery Systems (RVVDS)

As discussed in § 1, above, the transducer cell comprises a regulated viral vector delivery system (RVVDS). That is, the transducer cell genome is engineered so that it contains genes encoding viral proteins, whether forming a complete particle or a engineered version such as a VLP. A feature of the present invention is that activation of the RVVDS can be regulated so that viral transduction particles (VTPs) are not produced and released in the absence of a specific signal. Alternatively, VTP production is initially turned “on” and a signal (e.g., addition of a positive regulator or removal of a negative regulator is recognized by the RVVDS and terminates production. In various embodiments the regulated viral vector delivery system is activated by contact between the transducer cell and the target cell, by proximity of the transducer cell to the target cell, or by co-administration of the transducer cell and a chemical inducing agent (e.g., chemical drug, electrical, optical, magnetic, physical stimulus, etc.).

6.2.1 RVVDS Induced by Contact Between the Transducer Cell and the Target Cell

In one embodiment the RVVDS is induced when the transducer cell interacts (e.g., contacts) a target cell. Put differently, in one approach the inducing environment that results in RVVDS activation is proximity to or contact with the target cell. In one approach the contact may induce transcription from the first promoter through an engineered protein having an extracellular domain that binds or interacts with a cell surface molecule of a target cell and an intracellular signaling domain that mediates transcriptional activation. In one approach a synthetic Notch receptor (synNotch) is used. See Morsut et al., 2016, “Engineering customized cell sensing and response behaviors using synthetic notch receptors,” Cell 164:780-791; Lim et al., 2016, “Binding-Triggered Transcriptional Switches And Methods Of Use Thereof,” WO 2016/138034 (also published as US 20170233474), Gordley et al., 2016, “Modular Engineering of Cellular Signaling Proteins and Networks,” Current Opinion in Structural Biology 39: 106-114, each of which is incorporated herein by reference. In one approach, the synNotch receptor is a chimeric polypeptide comprising a) an extracellular domain comprising an antiligand that binds a cell surface molecule on a target cell, b) a Notch receptor polypeptide comprising one or more ligand-inducible proteolytic cleavage sites; and c) an intracellular domain comprising a transcription factor, wherein binding of the anti-ligand to the cell surface molecule induces cleavage of the Notch receptor polypeptide at the one or more ligand-inducible proteolytic cleavage sites, thereby releasing the transcription factor. In one approach, a chimeric antigen receptor T (CAR-T) cell is used. A CAR is a fusion protein combining an extracellular single chain antibody (scFv) with an intracellular regulatory domain of a T-cell receptor complex (Kalos et al., Sci Transl. Med., 2011:3). In another approach, cell contact (regardless of surface marker) can be used for regulation. Surface-bound CD43 paired with an intracellular CD45 has been shown to regulate transcriptional signaling pathways as demonstrated by Kojima et al., 2018, in “Nonimmune cells equipped with T-cell-receptor-like signaling for cancer cell ablation” Nat Chem Biol. 14(1):42-49, and Schukur et al. 2015, in “Implantable synthetic cytokine converter cells with AND-gate logic treat experimental psoriasis”, Sci Transl Med. 7(318):318ra201, demonstrating the variety of cell-contact based signaling pathways. TABLE 3 provides a examples, for illustration and not limitation, of surface-based receptor systems for RVVDS regulation.

TABLE 3

| | | | Expression regulatory |
|---|---|---|---|
| | | Transducer cell | element |
| Target cell | Transducer cell | intracellular | (e.g., transcription |
| surface protein | extracellular domain | signaling domain | factor/promoter) |
| CD19 | Anti-CD19 scFv | tTa (tetR-VP64) | TRE (tet responsive |
| | (SynNotch) | | promoter) |
| Cell contact with | CD43 | CD45 | Negatively regulates |
| any adjacent cell | | | JAK signaling |
| VEGF | VEGFR-1/VEGFR-2 | Tev-dCas9-VP64 | dCas9 driven by sgRNA |
| | | | selection to chosen site¹ |

¹See Baeumler et al., 2017, “Engineering Synthetic Signaling Pathways with Programmable dCas9-Based Chimeric Receptors” Cell Reports 20:2639-2653.

6.2.2 RVVDS Controlled by Coordinated Administration or Co-Administration of an Inducing Agent

In one approach, the promoter operably linked to the VTP is regulated by a small molecule. In this approach, transducer cells and the small molecule may be co-administered. Transducer cells may be administered to a patient and at a specific later time point, expression of the GMS may be altered. Examples of promoters induced by small molecules include “Tet-On Systems” for doxycycline-inducible expression (see, e.g., Das et al., 2016, “Tet-On Systems For Doxycycline-Inducible Gene Expression” Current Gene Therapy 16.3:156-167). In this context, co-administration does not necessarily mean simultaneous administration, but may refer to coordinated administration of the transducer cells at a first time point, followed by administration of an inducing agent at a later time point (e.g., 2, 5, 10, 20, 48, or 60 hours later, or later). In some embodiments, co-administration may be coordinated administration of an inducing agent followed by administration of transducer cells.

In one approach, the promoter operably linked to the viral genome is a constitutive human cell specific promoter. Constitutive human cell specific promoters include, but not limited to, human β-actin promoter (ACTB), elongation factor-1α (EF1α), phosphoglycerate kinase (PGK) and ubiquitin C (UbC) (Norman et al., (2010) PLoS ONE, 5(8):e12413). In some embodiments, the promoter operably linked to the viral genome is a human cell specific promoter such as, but not limited to, human CD4 promoter (Flamand et al., (1998) Journal of Virology, 72(11):8797-805 and Beil-Wagner et al., (2016) Scientific Reports, 6:21377). As noted previously, in some embodiments the promoter must also be a Pol-III promoter to successfully drive sgRNA expression for the use of some CRISPR systems as the GMS.

In some embodiments, the promoter operably linked to the viral genome is a tumor cell specific promoter such as, but not limited to, an alpha-fetoprotein (AFP) promoter, cholecystokinin-A receptor (CCKAR) promoter, carcinoembryonic antigen (CEA) promoter, c-erbB2 promoter, cyclo-oxygenase 2 isoform (COX-2) promoter, CXC-chemokine receptor 4 (CXCR4) promoter, mucin-like glycoprotein (MUC1) promoter, human epididymis protein 4 (HE4) promoter, and E2F1 transcription factor 1 (E2F-1) promoter. In some embodiments, the tumor cell specific promoter is active specifically in tumor cells such as, but not limited to, breast cancers, ovarian cancers, pancreatic cancers, prostate cancers, epithelial cancers, melanomas, and hepatocellular carcinomas.

In some embodiments, the target cell-specific promotor is chemically or physically altered. A chemically inducible promoter includes, but is not limited to, a promoter whose transcriptional activity is regulated by the presence or absence of alcohol (e.g., U.S. Pat. No. 9,434,953), tetracycline (e.g., Zabala et al., Cancer Research, (2004), 64:2799-2804 and U.S. Pat. No. 5,851,796), steroids (e.g., U.S. Pat. Nos. 5,512,483 and 6,784,340), metals (e.g., European Patent No: 0094428 B1), or other compounds (e.g., U.S. Pat. Nos. 9,388,425; 8,138,327 and U.S. Patent Publication 2012/0225933). In some embodiments, the target cell-specific promotor is chemically inducible by a small molecule, such as rapamycin or doxycycline (See, Bisht et al., (2017) Analytical Biochemistry, 530:40-49). For example, Rapamycin, can act as a chemical dimerizer causing the formation of a ternary complex between a first protein component (e.g., a plasma membrane anchored protein, such as FRB) and another protein component (e.g., FKBP) fused to a protein of interest (e.g., a cell surface receptor, such as CD25) freely diffusing in the target cell cytosol. As a result of the ternary complex, the protein of interest becomes localized and can induce expression of the GMS.

In some embodiments, the target cell-specific promotor is physically regulated. A physically inducible promoter includes, but is not limited to, a promoter whose transcriptional activity is regulated by the presence or absence of light (e.g., U.S. Pat. No. 5,750,385 and Published Patent Application No: 1998/040105), ionizing radiation (e.g., Weischelbaum et al. (1994) Cancer Research, 54:4266-4269; Hallahan et al. (1995) Nat Med., 1(8):786-791; Joki et al. (1995) Hum Gen Ther 6:1507-1513), or abnormal (e.g., low or high) temperatures (e.g., US Patent Application: 2003/0045495).

6.3. Promoters Controlling Expression in the Target Cell of GMS Components

In one approach, the promoter operably linked to the viral genome is a cell specific promoter. Examples of cell specific promoters include APOA2 (hepatocyte), IRSs (pancreatic beta cells), NPPA (ANF) cardiac, and TH (tyrosine hydrolase) CNS (dopaminergic neurons). For example, lentiviral vectors with tissue specific promoters are available from Flash Therapeutics (Toulouse, France) (flashtherapeutics.com). Other suitable promoters are known in the art. In one approach, the promoter operably linked to the viral genome is inducible be a small molecule.

6.4.Therapeutic Administration

A subject or patient in need of targeted genome modification (e.g., gene therapy) may be treated by administration of an effective amount of transducer cells, often by infusion or i.v., injection. The terms “treatment,” “treating,” and “treat” are defined as acting upon a disease, disorder, condition or genetic condition with an agent to reduce or ameliorate the effects of the disease, disorder, or condition and/or its symptoms.

The term “effective amount” or “therapeutically effective amount” as used herein refers to that amount of an embodiment of the agent (e.g., a compound, inhibitory agent, or drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the subject being treated has or is at risk of developing.

### 7. EXAMPLES

### 7.1 Example 1

This example we demonstrate that in a cell comprising a vector based on a replication defective adenovirus, replication is greater if E1A protein is provided in trans in the absence of E1B protein, compared to providing both E1A and E1B proteins.

We prepared a plasmid (“E1AB”) encoding the E1 gene (encoding E1A and E1B) and a plasmid (“E1A”) comprising a minimal gene encoding only E1A.

The “E1AB” plasmid included nucleotides 460 to 3533 of the Human adenovirus 5 genome (NCBI Reference Sequence: AC_000008.1). The 3074 n gene included the 5′ UTR, E1A exon1, intron, exon2 followed by E1B-19K, E1B-55K (E1B sequences overlap), 3′ UTR.

The “E1A” plasmid includes 870 n cDNA sequence of the E1A 32 kDa isoform. The cDNA sequence includes the only exons 1 and 2 of E1A and excludes all of E1B. The E1A protein sequence is provided above in Section 4.1.1.2. For this experiment, E1A was expressed as a fusion protein including monomeric Infrared Fluorescent Protein (mIFP) so that expression could be easily monitored

K562 cells were nucleofected with a negative control, E1A construct, or E1AB construct for 24 hours followed by mock or adenovirus infection (MOI 100) for 48 hours. Adenovirus DNA was extracted from cell supernatant and viral genomes were quantified via qPCR analysis. The fold change in adenovirus expression relative to the negative control is shown in Table 4 listed. (N=1)

TABLE 4

| | | | Fold |
|---|---|---|---|
| | | Adenovirus | change |
| | | genomes | in viral |
| | Infection | per ml | genomes |
| Sample | (MOI = 100) | supernatant | with E1 |
| negative control | mock | 4.21 | |
| negative control | AdΔE1Cre-GFP | 1.67E+06 | 1.00 |
| E1AB | mock | 1.33 | |
| E1AB | AdΔE1Cre-GFP | 5.34E+07 | 32.04 |
| E1A | mock | 2.12 | |
| E1A | AdΔE1Cre-GFP | 1.64E+08 | 98.48 |

### 7.2 Example 2

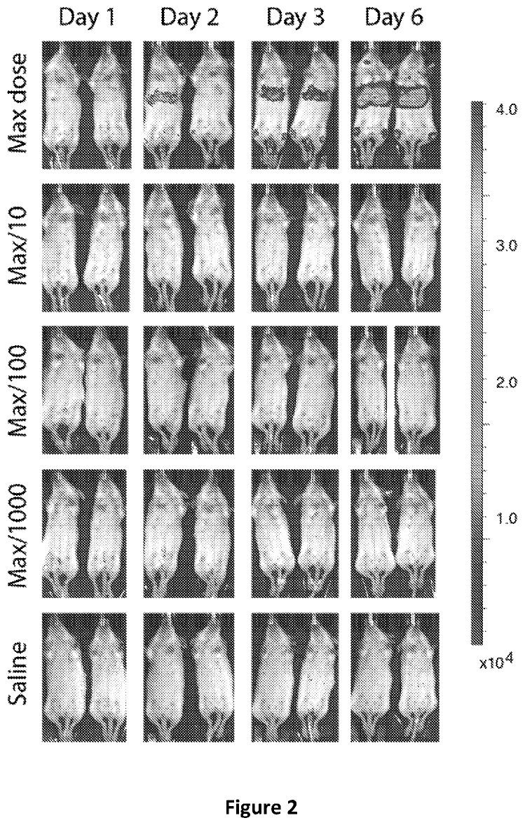

Empty transducer cells (lacking a RVVDS system) were engineered to constitutively express a luciferase protein for live, whole-body tracking of transducer cell motility. Multiple dilutions of RAW264.7 cells, starting at 2e6 cells diluted 10-fold up to 1,000, expressing firefly luciferase were administered by tail vein into B6 mice and monitored over a 6 day experiment to determine cellular motility. As demonstrated in FIG. 2, RAW264.7 cells are first seen in the tail vein, and are able to aggregate and reside in approximate liver and lymph node tissues. Further dilutions showed little to no signal because of poor firefly luciferase production.

### 7.3 Example 3

This prophetic example describes using isolated autologous transducer cells for in vivo editing of the dystrophin (DMD) gene in muscle cells of a patient with Duchene Muscular Dystrophy. The patient has a pathogenic frameshift mutation in DMD resulting from the deletion of the nucleotides “CAAA” at positions 9204-9207 (Taylor et al., 2007).

### 1. VIRAL VECTOR

An adenoviral viral vector based on the pAdEasy1 plasmid (He, Zhou, Da Costa, Kinzler, & Vogelstein, 1998; Luo et al., 2007) is used. The vector contains a DNA sequence encoding Streptococcus pyogenes Cas9 system (Mali et al., 2013) under control of the EF1αconstitutive promoter (Matsuda & Cepko, 2004). Cas9, pAdEasy1, and the EF1αpromoter sequences are available from the nonprofit plasmid repository Addgene (addgene.org/crispr/church/; addgene.org/16400/, and addgene.org/11154/, respectively). The vector also includes a DNA sequence encoding a targeting sgRNA for directing genome editing in a target cell to across positions 9204-9207 in the DMD gene, where insertion of the CAAA (back to wild-type) prevents further targeting. The sgRNA is under transcriptional control of the U6 promoter (Cong et al., 2013). The vector also includes an approximately 800 bp stretch of repair DNA sequence from the wildtype DMD sequence (NCBI Reference Sequence: NG_012232.1) (Koenig et al., 1988). This sequence repairs the DNA break following cleavage by Cas9, reintroducing the nucleotides CAAA to positions 9204-9207, and thereby repairing the frameshift mutation. pAdEasy1 lacks the viral genes E1 and E3, and the EF1α-Cas9, U6-sgRNA, and repair DNA sequences are inserted with the available packaging adenovirus capacity.

### 2. CELLS

T lymphocytes are recovered from peripheral blood of the patient and modified ex vivo to contain components of the packaged viral delivery system including: E1 proteins under the control of a doxycycline inducible promoter, the rtTA activator domain expressed from a constitutive plasmid (Das et al., 1992), and the transduction with the adenoviral vector described above.

### 3. INFUSION AND INDUCTION

After ex vivo manipulation the cells are re-infused into the patient, using the method described in Kochenderfer et al., 2010. Doxycycline (100 mg) is administered orally to the patient twice per day for 3 days.

### 4. EVALUATION

A muscle biopsy from the gastrocnemius muscle indicates that successful gene editing occurred in >10% of skeletal muscle cells.

### 5. REFERENCES FOR EXAMPLE 3

Cong, L., Ran, F. A., Cox, D., Lin, S., Barretto, R., Habib, N., et al. (2013). Multiplex genome engineering using CRISPR/Cas systems. Science, 339(6121), 819-823. doi.org/10.1126/science.1231143; Das, A. T., Tenenbaum, L., & Berkhout, B. (2016). Tet-On Systems For Doxycycline-inducible Gene Expression. Current Gene Therapy, 16(3), 156-167. doi.org/10.2174/1566523216666160524144041; Gossen, M., & Bujard, H. (1992). Tight control of gene expression in mammalian cells by tetracycline-responsive promoters. Proceedings of the National Academy of Sciences of the United States of America, 89(12), 5547-5551; He, T.-C., Zhou, S., Da Costa, L. T., Kinzler, K. W., & Vogelstein, B. (1998). A simplified system for generating recombinant adenoviruses. Proceedings of the National Academy of Sciences of the United States of America, 1-6; Kochenderfer, J. N., Wilson, W. H., Janik, J. E., Dudley, M. E., Stetler-Stevenson, M., Feldman, S. A., et al. (2010). Eradication of B-lineage cells and regression of lymphoma in a patient treated with autologous T cells genetically engineered to recognize CD19. Blood, 116(20), 4099-4102. doi.org/10.1182/blood-2010-04-281931; Koenig, M., Monaco, A. P., & Kunkel, L. M. (1988). The complete sequence of dystrophin predicts a rod-shaped cytoskeletal protein. Cell, 53(2), 219-228. Retrieved from eutils.ncbi.nlm.nih.gov/entrez/eutils/elink.fcgi?dbfrom=pubmed&id=3282674&retmode=ref&cmd=p rlinks; Luo, J., Deng, Z.-L., Luo, X., Tang, N., Song, W.-X., Chen, J., et al. (2007). A protocol for rapid generation of recombinant adenoviruses using the AdEasy system. Nature Protocols, 2(5), 1236-1247. doi.org/10.1038/nprot.2007.135; Mali, P., Yang, L., Esvelt, K. M., Aach, J., Guell, M., DiCarlo, J. E., et al. (2013). RNA-guided human genome engineering via Cas9. Science, 339(6121), 823-826. doi.org/10.1126/science.1232033; Matsuda, T., & Cepko, C. L. (2004). Electroporation and RNA interference in the rodent retina in vivo and in vitro. Proceedings of the National Academy of Sciences of the United States of America, 101(1), 16-22. doi.org/10.1073/pnas.2235688100; Taylor, P. J., Maroulis, S., Mullan, G. L., Pedersen, R. L., Baumli, A., Elakis, G., et al. (2007). Measurement of the clinical utility of a combined mutation detection protocol in carriers of Duchenne and Becker muscular dystrophy. Journal of Medical Genetics, 44(6), 368-372. doi.org/10.1136/jmg.2006.047464;

### 7.4 Example 4

This prophetic example describes using a mouse macrophage cell line as a transducer cell for in vivo activation of a Cre-lox mediated luciferase reporter in hypoxic regions of a mouse. The mouse (Gt(ROSA)26Sor^{tm1(Luc)Koel}) contains DNA encoding the firefly luciferase (luc) gene inserted into the Gt(ROSA)26Sor locus (Safran et al., 2003). Luciferase is not expressed because of a loxP-flanked STOP fragment between the luc coding sequence and the Gt(ROSA)26Sor promoter. A lentiviral vector was used to transduce cells in vivo with Cre-recombinase only under hypoxic conditions, and thus remove the STOP fragment, bringing luciferase under control of the constitutively expressed Gt(ROSA)26Sor promoter and only expressing in hypoxic regions of the mouse after Cre delivery.

### 1. VIRAL VECTOR

A third-generation lentiviral vector system is used (Sakuma, Barry, & Ikeda, 2012). A hypoxia-regulated element (“5HRE”) is used to promote transcription of components of the viral packaging system so that they will only be transcribed only in hypoxic environments (Vordermark, Shibata, & Brown, 2001). The lentiviral vector system comprised multiple plasmids including an envelope plasmid containing a DNA sequence encoding the envelope protein VSV-G transcriptionally regulated by 5HRE, a packaging plasmid containing DNA sequences encoding gag and pol transcriptionally regulated by 5HRE, and a second packaging plasmid containing a DNA sequence encoding rev transcriptionally regulated by 5HRE. In addition, there is a transfer plasmid containing a DNA sequence encoding Cre under the control of constitutively active promoter EF1α, and flanked by two LTR sequences (Matsuda & Cepko, 2004). Lentiviral envelope, packaging, and transfer plasmids, EF1α, 5HRE, and Cre sequences are available from the nonprofit plasmid repository Addgene (addgene.org/viral-vectors/lentivirus/; addgene.org/11154/; addgene.org/46926/; and addgene.org/49056/; respectively).

### 2. CELLS

An immortalized mouse macrophage cell line (RAW264.7) is transfected with the viral vector delivery system plasmids by electroporation (Raschke, Baird, Ralph, & Nakoinz, 1978; Smale, 2010).

### 3. INFUSION

After transfection, the macrophages are introduced into the Gt(ROSA)26Sor^{tm1(LUC)Kae1} mouse by injection via the tail vein (Weisser, van Rooijen, & Sly, 2012).

### 4. EVALUATION

In vivo bioluminescence imaging scans show the activation of luciferase in hypoxic regions of the mouse (Zinn et al., 2008).

### 5. REFERENCES FOR EXAMPLE 2

Matsuda, T., & Cepko, C. L. (2004). Electroporation and RNA interference in the rodent retina in vivo and in vitro. Proceedings of the National Academy of Sciences of the United States of America, 101(1), 16-22. doi.org/10.1073/pnas.2235688100; Raschke, W. C., Baird, S., Ralph, P., & Nakoinz, I. (1978). Functional macrophage cell lines transformed by Abelson leukemia virus. Cell, 15(1), 261-267; Safran, M., Kim, W. Y., Kung, A. L., Horner, J. W., DePinho, R. A., & Kaelin, W. G. (2003). Mouse reporter strain for noninvasive bioluminescent imaging of cells that have undergone Cre-mediated recombination. Molecular Imaging, 2(4), 297-302; Sakuma, T., Barry, M. A., & Ikeda, Y. (2012). Lentiviral vectors: basic to translational. Biochemical Journal, 443(3), 603-618. doi.org/10.1042/BJ20120146; Smale, S. T. (2010). Transfection by electroporation of RAW 264.7 macrophages. Cold Spring Harbor Protocols, 2010(2), pdb.prot5374-pdb.prot5374. doi.org/10.1101/pdb.prot5374; Vordermark, D., Shibata, T., & Brown, J. M. (2001). Green fluorescent protein is a suitable reporter of tumor hypoxia despite an oxygen requirement for chromophore formation. Neoplasia (New York, N.Y.), 3(6), 527-534. doi.org/10.1038/sj/neo/7900192; Weisser, S. B., van Rooijen, N., & Sly, L. M. (2012). Depletion and reconstitution of macrophages in mice. Journal of Visualized Experiments: JoVE, (66), 4105-e4105. doi.org/10.3791/4105; Zinn, K. R., Chaudhuri, T. R., Szafran, A. A., O'Quinn, D., Weaver, C., Dugger, K., et al. (2008). Noninvasive bioluminescence imaging in small animals. ILAR Journal, 49(1), 103-115;

### 7.5 Example 5

This prophetic example describes using an isolated autologous transducer cell for in vivo editing of the HTT gene in the brain of a patient with Huntington's Disease. Huntington's Disease is associated with an expansion of the CAG trinucleotide sequence in exon 1 of HTT (Macdonald et al., 1993; Sturrock & Leavitt, 2010). A healthy HTT gene is defined as having between approximately 10 and 35 CAG repeats. In the HTT alleles in a patient with Huntington's disease, the number of repeats is expanded to above this range (typically 36-60).

### 1. VIRAL VECTOR

A lentiviral vector constructed from the murine leukemia virus (MLV) is used as the viral vector packaging system. MLV has been shown to transduce brain tumor cells in humans in vivo, and lentiviral vectors have successfully transduced nonhuman primate and rat neurons in vivo (Kittler, Moss, Osten, Dittgen, & Licznerski, 2006; Lee et al., 2001). The lentiviral vector system comprises multiple plasmids including an envelope plasmid containing a DNA sequence encoding the envelope protein VSV-G that was transcriptionally regulated by 5× Gal4-responsive elements, a packaging plasmid containing DNA sequences encoding gag and pol that are transcriptionally regulated by 5× Gal4-responsive elements, and a second packaging plasmid containing a DNA sequence encoding rev that is transcriptionally regulated by 5× Gal4-responsive elements (Roybal:2016ds; Griggs & Johnston, 1991). In addition, there is a transfer plasmid containing a DNA sequence encoding two ZFN proteins under control of the neural cell-specific for Gpr88, where the DNA-binding domain for each zinc finger targets the CAG repeats in exon 1 of HTT, all flanked by two LTR sequences (Cong et al., 2013; Hisatsune, Ogawa, & Mikoshiba, 2013; Mali et al., 2013). After cleavage, larger CAG repeats collapsed down to within the healthy range of 10-35 repetitive elements.

### 2. CELLS

Macrophages are recovered from peripheral blood of the patient and modified ex vivo to contain components of the packaged viral delivery system including DNA encoding a synthetic Notch receptor to the brain-specific G-protein coupled receptor GPR85 (α-GPR85 synNotch) (Roybal et al., 2016). The α-GPR85 synNotch receptor further comprises an intracellular Gal4 DNA-binding domain fused to a viral transcriptional activator domain (VP64) (Roybal et al., 2016). The macrophages are transfected with the lentiviral vector described above.

### 3. INFUSION AND INDUCTION

After ex vivo manipulation the cells are re-infused into the patient, using the method described in (Fraser et al., 2017). Macrophages are known to cross the blood brain barrier, and the blood brain barrier is impaired in Huntington's disease (Drouin-Ouellet et al., 2015; Fitch & Silver, 1997)

### 4. EVALUATION

A brain biopsy and PCR-based genetic diagnostic test indicates that successful gene editing occurred in >12% of brain cells (Goldberg, et al., 1994).

### 5. REFERENCES

References for Example 3; Cong, L., Ran, F. A., Cox, D., Lin, S., Barretto, R., Habib, N., et al. (2013). Multiplex genome engineering using CRISPR/Cas systems. Science, 339(6121), 819-823; Drouin-Ouellet, J., Sawiak, S. J., Cisbani, G., Lagacé, M., Kuan, W.-L., Saint-Pierre, M., et al. (2015). Cerebrovascular and blood-brain barrier impairments in Huntington's disease: Potential implications for its pathophysiology. Annals of Neurology, 78(2), 160-177; Fitch, M. T., & Silver, J. (1997). Activated Macrophages and the Blood-Brain Barrier: Inflammation after CNS Injury Leads to Increases in Putative Inhibitory Molecules. Experimental Neurology, 148(2), 587-603; Fraser, A. R., Pass, C., Burgoyne, P., Atkinson, A., Bailey, L., Laurie, A., et al. (2017). Development, functional characterization and validation of methodology for GMP-compliant manufacture of phagocytic macrophages: A novel cellular therapeutic for liver cirrhosis. Cytotherapy, 19(9), 1113-1124; Goldberg, Y. P., Andrew, S. E., Clarke, L. A., & Hayden, M. R. (1993). A PCR method for accurate assessment of trinucleotide repeat expansion in Huntington disease. Human Molecular Genetics, 2(6), 635-636; Griggs, D. W., & Johnston, M. (1991). Regulated expression of the GAL4 activator gene in yeast provides a sensitive genetic switch for glucose repression., 88(19), 8597-8601; Hisatsune, C., Ogawa, N., & Mikoshiba, K. (2013). Striatum-specific expression of Cre recombinase using the Gpr88 promoter in mice. Transgenic Research, 22(6), 1241-1247; Kittler, J. T., Moss, S. J., Osten, P., Dittgen, T., & Licznerski, P. (2006). Lentivirus-based genetic manipulations in neurons in vivo; Kremer, B., Goldberg, P., Andrew, S. E., Theilmann, J., Telenius, H., Zeisler, J., et al. (1994). A worldwide study of the Huntington's disease mutation. The sensitivity and specificity of measuring CAG repeats. The New England Journal of Medicine, 330(20), 1401-1406; Lee, H., Song, J. J., Kim, E., Yun, C. O., Choi, J., Lee, B., et al. (2001). Efficient gene transfer of VSV-G pseudotyped retroviral vector to human brain tumor. Gene Therapy, 8(4), 268-273; Macdonald, M., et al. (1993). A novel gene containing a trinucleotide repeat that is expanded and unstable on Huntington's disease chromosomes. Cell, 72(6), 971-983; Mali, P., Yang, L., Esvelt, K. M., Aach, J., Guell, M., DiCarlo, J. E., et al. (2013). RNA-guided human genome engineering via Cas9. Science, 339(6121), 823-826; Roybal, K. T., Williams, J. Z., Morsut, L., Rupp, L. J., Kolinko, I., Choe, J. H., et al. (2016). Engineering T Cells with Customized Therapeutic Response Programs Using Synthetic Notch Receptors. Cell, 167(2), 419-432.e16; Sturrock, A., & Leavitt, B. R. (2010). The Clinical and Genetic Features of Huntington Disease. Journal of Geriatric Psychiatry and Neurology, 23(4), 243-259.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. An isolated mammalian transducer cell comprising:
a regulated viral vector delivery system (RVVDS) for producing and releasing viral transduction particles (VTPs) when the transducer cell is exposed to inducing conditions;
wherein each VTP comprises a nucleic acid encoding a genome modification system (GMS) comprising a genome modification protein and one or more elements that regulate expression or activity of the genome modification protein in a mammalian cell.

2. The transducer cell of claim 1 wherein the RVVDS comprises one or more first promoters that control VTP production and release, wherein at least one first promoter is an inducible promoter, and the one or more elements that regulate expression or activity of the genome modification protein comprise one or more second promoters, wherein at least one second promoter is an inducible and/or cell specific promoter.

3. The transducer cell of claim 2 wherein the RVVDS comprises a first vector that is a replication deficient adenovirus vector comprising the GMS and a second vector comprising a nucleic acid sequence encoding an adenovirus E1A protein.

4. The transducer cell of claim 3 wherein the first vector comprises a first first promoter and the second vector comprises a second first promoter.

5. The transducer cell of claim 4 wherein the nucleic acid sequence encoding an adenovirus E1A protein does not include an intron(s).

6. The transducer cell of claim 5 that does not comprise a gene sequence encoding an adenovirus E1B protein.

7. The transducer cell of claim 6 wherein the adenovirus E1A protein is a human adenovirus 5 E1A protein comprising an amino acid sequence of SEQ ID NO:1.

8. The transducer cell of claim 1 wherein the genome modification protein is a nuclease.

9. The transducer cell of claim 8 wherein the nuclease is selected from the group consisting of a CRISPR-associated protein (Cas), a Cre recombinase, a zinc-finger nuclease (ZFN), and a transcription activator-like effector nuclease (TALEN).

10. The transducer cell of claim 8 wherein the GMS comprises an RNA-guided nuclease and the VTP comprises a sequence encoding a nucleic acid targeting moiety.

11. The transducer cell of claim 9 wherein the nuclease is a CRISPR-associated protein (Cas) and the nucleic acid targeting moiety is a guide RNA.

12. The transducer cell of claim 11 wherein the Cas is Cas9 and the ZFN is a Fok1 fusion protein.

13. (canceled)

14. The transducer cell of claim 2 wherein the at least one second promoter comprises a cell-specific promoter and/or an inducible promoter.

15. The transducer cell of claim 14 wherein the at least one second promoter comprises a doxycycline-inducible promoter.

16. The transducer cell of claim 2 wherein the inducing conditions are proximity to a target cell comprising a cell-surface molecule, and activation of at least one first promoter is mediated by an engineered protein in the transducer cell, said engineered protein having an extracellular domain that interacts with a cell surface molecule of a target cell and an intracellular signaling domain that activates transcriptional activation from the at least one first promoter in the transducer cell, or the inducing conditions are exposure of the transducer cell to a small molecule transcriptional activator that activates transcription from at least one first promoter.

17. (canceled)

18. The transducer cell of claim 1 that is a macrophage, lymphocyte, T cell, NK cell, B cell, plasma cell, dendritic cell, neutrophil, eosinophil, basophil, monocyte, or stem cell or an engineered human cell.

19. (canceled)

20. An in vivo method of modifying a genome of a target cell in a mammalian subject, said method comprising administering an effective amount of the transducer cell of claim 1 to the subject.

21. An in vivo method of modifying a genome of a target cell in a mammalian subject, said method comprising administering an effective amount of the transducer cell of claim 2 wherein the regulated viral vector delivery system is activated by contact between the transducer cell and the target cell, by proximity of the transducer cell to the target cell, or by co-administration of the transducer cell and an regulating agent to the subject,
wherein the second promoter is active, or becomes active, in the target cell.

22. The method of claim 21 wherein the VTP comprises a nucleic acid targeting sequence with homology to a target nucleic acid sequence in the genome of the target cell.

23. A mammalian cell that comprises a first polynucleotide encoding a replication defective adenovirus vector comprising a genetic cargo and a second polynucleotide that encodes an Adenovirus E1A protein with the proviso that the cell does not comprise an Adenovirus E1B protein or a polynucleotide that encodes an adenovirus E1B protein.
